(19) United States
(12) Patent Application Publication
(10) Pub. No.: US 2024/0016877 A1
(43) Pub. Date: Jan. 18, 2024

(54) ANTIBIOTIC COMPOSITION AND METHOD OF MANUFACTURING THE SAME

(71) Applicant: MINH TAM THI TRAN, HO CHI MINH (VN)

(72) Inventor: MINH TAM THI TRAN, HO CHI MINH (VN)

(21) Appl. No.: 17/933,484

(22) Filed: Sep. 20, 2022

(30) Foreign Application Priority Data
Jul. 12, 2022 (VN) 1-2022-04383

(51) Int. Cl. A61K 36/8962; A61K 36/9068; A61K 36/67; A61K 36/31; A61K 36/73; A61K 36/9066; A61K 36/54; A61K 31/12; A61P 31/04

(52) U.S. Cl. CPC A61K 36/8962; A61K 36/9068; A61K 36/67; A61K 36/31; A61K 36/73; A61K 36/9066; A61K 36/54; A61K 31/12; A61P 31/04

(57) ABSTRACT

An antibiotic composition and a method of preparing the same are disclosed which comprises: a mixture of liquid fruits and spices having antimicrobial properties at predetermined concentration ratio, all undergone an anoxic fermentation process without being exposed to sunlight for a predetermined period of time and at a predetermined temperature so that the antibiotic composition is characterized by having antimicrobial activities and polyphenol bioavailability.

### CLAIM OF PRIORITY

This application claims priority under 35 U.S.C. § 112(d) of Application No. 1-2022-04383, filed on Jul. 12, 2022 (Jul. 12th 2022), in the Republic Socialist of Vietnam, entitled, “Ché̊phà̊m kháng sinh và phuong pháp tao ché̊ phà̊m kháng sinh này”. The patent application identified above is incorporated here by reference in its entirety.

### FIELD OF THE INVENTION

The present invention relates generally to the field of antibiotic beverages. More specifically, the present invention relates to an antibiotic composition capable of enhancing the bioavailability of antimicrobial and at the same time weakening antimicrobial resistance (AMR) mechanism of bacteria and fungi.

### BACKGROUND ART

Since the ancient time, humans have used antibiotics to fight various pathogenic bacteria and fungi. Antibiotics—also known as antibacterials are medications that destroy or slow down the growth of bacteria—are in form of either liquid beverages, pills, ointment, creams, prescription or over the counter (OTC) non-prescription medicines. Man-made antibiotics such as penicillin and natural antibiotic such as garlic or curcumine are powerful medicines proven to fight certain diseases and save lives if used properly. Antibiotics can treat different diseases such as fungal infections, syphilis, whooping coughs, and diphtheria, etc. In addition, antibiotics can be prescribed prophylactically to prevent post-surgery infections. In different situations, HIV/AIDS or otherwise immunosuppressed patients may be prescribed antibiotics to prevent secondary bacterial infections.¹ This is also true in the case of cancer patients with suppressed immune systems, patients about to undergo high risk surgeries, as well as dental patients at risk for bacterial endocarditis.² Even though antibiotics are not effective against viruses such as upper respiratory tract infections (URTI) such as common cold and flu, they can work in concert with the body defense mechanism to boost the immune system. Thus, antibiotics are proven effective in curing and preventing diseases, and/or boosting human immune system.

However, antimicrobial resistance (AMR) or the ability of bacteria and fungi to develop resistance to antibiotics have been noticed due to the overuses of antibiotics. That is, bacteria and fungi can become more and more intrinsically resistant to antibiotics, rendering these antibiotics ineffective. Antibiotic resistance is a naturally occurring process to fungi and bacteria. However, increases in antibiotic resistance are driven by a combination of germs exposed to antibiotics, and the spread of those germs and their resistance mechanisms. At least twenty three thousands (23,000) people die each year as a direct result of these antibiotic-resistant infections. Almost 250,000 people each year need hospital cares for treatment of Clostridium difficile (C. difficile) infections. This infection is very difficult to treat. Gram-negative bacteria have an outer layer (membrane) that protects them from their environment. These bacteria can use this membrane to selectively keep antibiotic drugs from entering. Some Pseudomonas aeruginosa bacteria can produce pumps to get rid of several different important antibiotic drugs, including fluoroquinolones, beta-lactams, chloramphenicol, and trimethoprim. Klebsiella pneumoniae bacteria produce enzymes called carbapenemases, which break down carbapenem drugs and most other beta-lactam drugs. Escherichia coli bacteria with the mcr-1 gene can add a compound to the outside of the cell wall so that the drug colistin cannot latch onto it. Some Staphylococcus aureus bacteria can bypass the drug effects of trimethoprim. Thus, there exists a need to develop more potent antibiotics that can not only kill bacteria but also prevent them from developing AMR. This has led to an increased interest in medicinal plants since 25-50% of current pharmaceuticals are plant derived. Plant extracts have the ability to bind to protein domains leading to modification or inhibition protein-protein interactions. This enables the herbals to present themselves as effective modulators of host related cellular processes viz immune response, mitosis, apoptosis and signal transduction. Thus they may exert their activity not only by killing the microorganism but by affecting key events in the pathogenic process, thereby, the bacteria, fungi and viruses may have a reduced ability to develop resistance to botanicals.

Now referring to FIG. 1, a diagram 100 of an evolution of various plant based antibiotics in term of the diameter of inhibition zone (DIZ) of is illustrated. DIZ measures how effective a particular antibiotic fight against certain bacteria. That is, the larger the DIZ the better that particular antibiotic kill and/or stop the growth of bacteria or fungi. The DIZ is obtained from the Kirby-Bauer test and it is well known in the art. Therefore, the DIZ test is not described in details herein. Summarily, this test takes a pure bacterial culture and spreads it onto an agar plate. Then, a small disk infused with antibiotics (appropriately called an antibiotic disk) is placed onto the agar plate. Various disks with different antibiotics are placed around the plate, and the bacteria are left to incubate for a certain amount of time. Once the disk is placed onto the plate, the antibiotics will start to diffuse out. If the bacteria being studied is sensitive to the antibiotic, then no bacteria will grow close to the disk because it will be killed by the medication. But farther away from the antibiotic disk, the concentration of the antibiotic decreases. At a certain distance from the disk, the bacterial grow again because the antibiotic concentration is too low to affect the bacteria. The area around the antibiotic disk that has no bacterial growth is known as the zone of inhibition (DIZ). The zone of inhibition is a uniformly circular zone of no bacterial growth around the antibiotic disk. The larger this zone is, the more sensitive the bacteria is to that antibiotic. The smaller the zone is, the more resistant (and, thus, less sensitive) the bacteria is.

Continuing with FIG. 1, a first antibiotic sample 101 is a single material extracted from natural resources. It is finely ground and dried (dehydrated). An example of a first antibiotic sample 101 is garlic or curcumin. A second antibiotic sample 102 is a mixture of natural medicinal plants such as garlic, ginger, and lemon grass that are also finely ground and dried. As seen, second antibiotic sample 102 has a larger zone of inhibition (DIZ) than that of first antibiotic sample 101. Both first antibiotic sample 101 and second antibiotic sample 102 are well-known herbal medicines. It is noted that a third antibiotic sample 103, a fourth antibiotic sample 104, and a fifth antibiotic sample 105 are developed by the inventor of the present invention. Third antibiotic sample 103 contains mixed materials forming sauerkraut which was undergone anerobic fermentation to produce Lactobacillus sp. Mixed materials were minced and then mixed with apple vinegar. This mixture was fermented anoxically to form antibiotic sample 104 which is a minced fermentation. This minced fermentation method gives fourth antibiotic sample 104 a DIZ greater than that of third antibiotic sample 103. In the next antimicrobial level, selected medicinal plants are juiced and then mixed with apple vinegar. The mixture is undergone an anoxic fermentation process to form fifth antibiotic sample 105. This process is called juiced fermentation that gives fifth antibiotic sample 105 a larger DIZ than that of fourth antibiotic sample 104 which is larger than that of third antibiotic sample 103.

Next, referring to FIG. 2, a plot 200 of DIZ versus different prior-art antibiotic samples including antibiotic samples 103-105 is illustrated. The Kirby-Bauer test was performed for a Bragg apple cider vinegar (ACV) 201, a material juice 202, a third antibiotic sample 203, a fourth antibiotic sample 204, and a fifth antibiotic sample 205 against various bacteria such as S. aureus, K. pneumonia, E. coli, P. aeruginosa, Salmonella sp., and Listeria monocytogenes. Bragg ACV 201 is original wellness elixir from organic apple manufactured by the Bragg® Company. Material juice 202 is made from various organic fruit juice such as garlic, ginger, and salt. Third antibiotic sample 203, fourth antibiotic sample 204, and fifth antibiotic sample 205 are the same as third antibiotic sample 103, fourth antibiotic sample 104, and fifth antibiotic sample 105 respectively—which are all previous inventions by the present inventor. The tests showed that Bragg AVC 201 has a diameter of inhibition zone (DIZ) less than 12.5 mm against S. aureus, K. pneumonia, E. coli, and P. aeruginosa. Material juice 202 has a DIZ less than 12.5 mm while third antibiotic sample 203 and fourth antibiotic sample 204 have higher DIZs about 13 mm and fifth antibiotic sample 205 have a DIZ about 14 mm against the same group of bacteria S. aureus, K. pneumonia, E. coli, P. aeruginosa, Salmonella sp., and Listeria monocytogenes. Thus, from the above experiment, it was learned that the selected materials, their percentage weight or volume (% w/w or % v/v), and the process of fabrication contribute to the DIZ and the effectiveness against bacteria and fungi.

Therefore what is needed is a medicinal plant antibiotic composition and process that are simple and inexpensive to make. What is needed is method that produces an antibiotic that is effective against different bacteria and fungi (larger DIZ), comprised of polyphenols capable of preventing bacteria from developing antimicrobial resistance (AMR). What is needed is an antibiotic composition that is easy to absorb by humans.

The present invention solves the above described problems and meets all of the above needs.

### SUMMARY OF THE INVENTION

Accordingly, an object of the present invention is to provide an antibiotic composition capable of preventing bacteria and fungi from forming antimicrobial resistance (AMR).

Another object of the present invention is to provide an antibiotic composition having strong antibacterial capabilities (DIZ>15 mm) in comparison to existing antibiotics available in the market.

Another object of the present invention is to provide an antibiotic composition that is synergetically working with other antibiotics and the immune system to fight against bacteria and fungi.

Another object of the present invention is to provide a simple and inexpensive method of making an antibiotic tonic.

Another object of the present invention is to provide a method of making an antibiotic composition comprising (a) selecting various antimicrobial fruits having equal first predetermined percentage weight or volume (% w/w or % v/v) of 1:1 to one another; (b) juicing the selected antimicrobial fruits to obtain antimicrobial fruit juice; (c) mixing the antimicrobial fruit juice with a second solvent juice having equal second predetermined weight or volume (% w/w or % v/v) with the antimicrobial fruit juice; (d) adding a first powder spice, a second powder spice having a third predetermined percentage weight or volume (% w/w or % v/v), and a third powder spice having a fourth predetermined percentage weight or volume (% w/w or % v/v) with respect to the antimicrobial fruit juice and the AVC to form a solution; and (d) anoxic fermenting and simultaneously stirring the solution in an absence of light condition for a predetermined period of time and for a predetermined temperature range.

Yet another object of the present invention is to provide an antibiotic composition and a method of preparing the same are disclosed which comprises: a mixture of liquid fruits and spices having antimicrobial properties having predetermined weight (% w/w) or volume ratio (% v/v), all undergone an anoxic fermentation process without being exposed to sunlight for a predetermined period of time and at a predetermined temperature so that the antibiotic composition is characterized by having a high antimicrobial activities and polyphenol bioavailability.

These and other advantages of the present invention will no doubt become obvious to those of ordinary skill in the art after having read the following detailed description of the preferred embodiments, which are illustrated in the various drawing figures.

### BRIEF DESCRIPTION OF THE DRAWINGS

The accompanying drawings, which are incorporated in and form a part of this specification, illustrate embodiments of the invention and, together with the description, serve to explain the principles of the invention.

FIG. 1 illustrates a development of five different prior-art antibiotics that have increasing diameter of inhibition (DIZ);

FIG. 2 is a plot describing the DIZ of five different prior-art antibiotics against different groups of bacteria and fungi;

FIG. 3 is a flowchart illustrating a process of manufacturing an antibiotic compound in accordance with an exemplary embodiment of the present invention;

FIG. 4 illustrates different composition and their percentage weights (% w/w or % v/v) in the implementation of the process 300 in accordance with an exemplary embodiment of the present invention.

FIG. 5 is plot describing the DIZ of the antibiotic compound of the present invention against the different bacteria and fungi.

FIG. 6 is a comparative plots identifying curcumin content (mg/g) of different antibiotics including the antibiotic composition of the present invention.

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well-known methods, procedures, components, and circuits have not been described in detail so as not to unnecessarily obscure aspects of the present invention.

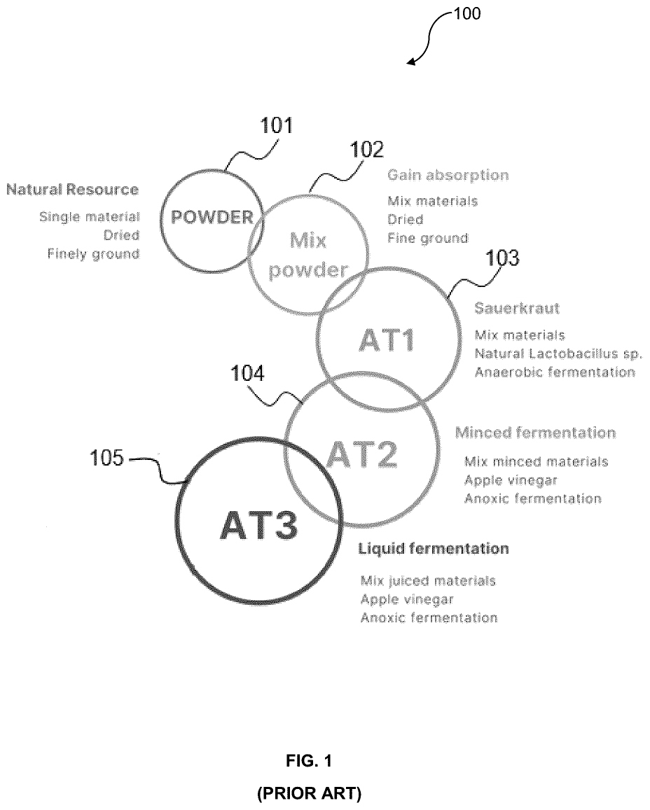

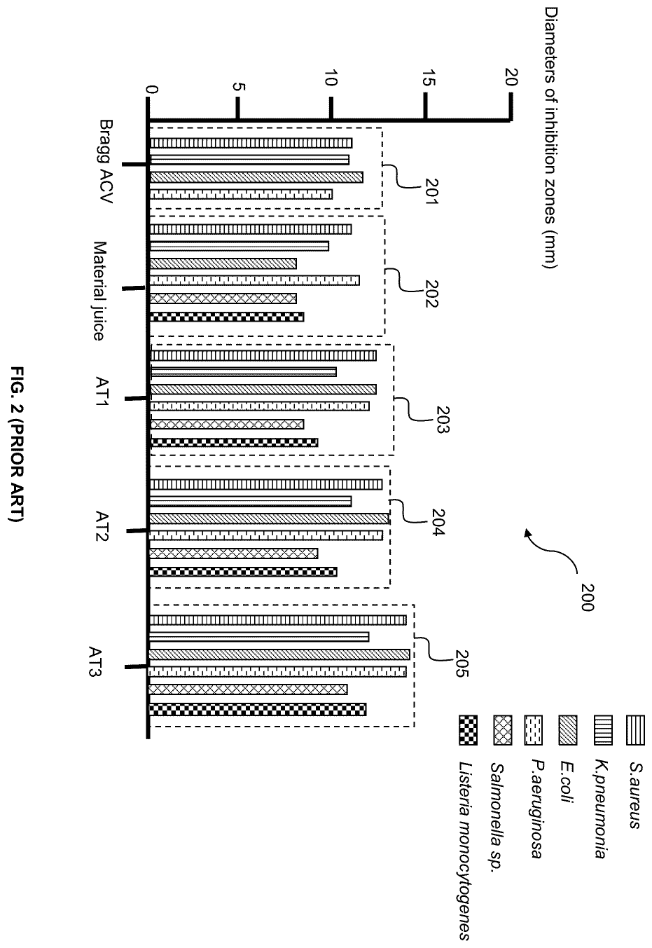

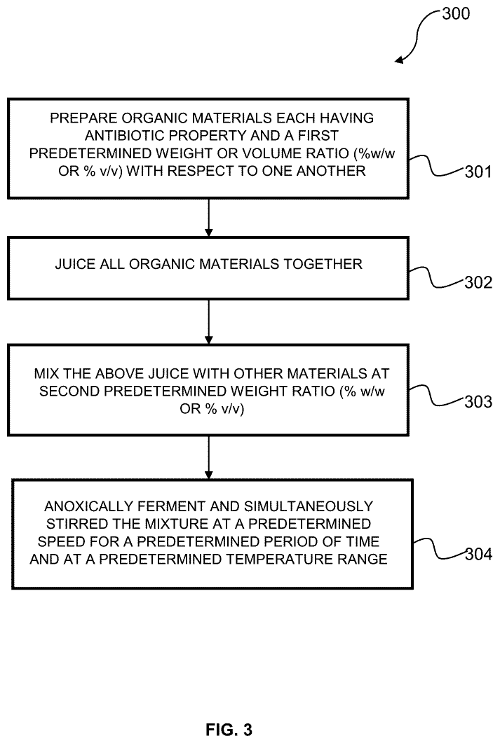

One embodiment of the invention is now described with reference to FIG. 3. FIG. 3 is a flowchart 300 illustrating a process of manufacturing an antibiotic compound in accordance with an exemplary embodiment of the present invention. Upon the completion of the following steps, an antibiotic compound is achieved that attains all the objects of the present invention such as gaining in bio-absorption, effectiveness against different groups of bacteria and fungi (having larger DIZ as compared to other antibiotic beverages described in FIG. 1 and FIG. 2), and polyphenols capable of preventing bacteria from developing antimicrobial resistance (AMR).

At step 301, different organic materials having relative percentage weight or volume (% w/w or % v/v) are selected for their antimicrobial properties. In one aspect of the present invention, a group of medicinal plants or herbs with known antimicrobial properties including a garlic having a predetermined first percentage weight or volume (% w/w or % v/v); a ginger having a predetermined second percentage weight or volume (% w/w or % v/v); an onion having a predetermined third percentage weight or volume (% w/w or % v/v); a garlangal root having a predetermined fourth percentage weight or volume (% w/w or % v/v); a belly pepper having a predetermined fifth percentage weight or volume (% w/w or % v/v); a turmeric having a predetermined sixth percentage weight or volume (% w/w or % v/v); and a raddish having a predetermined seventh percentage weight or volume (% w/w or % v/v) are selected. In one aspect of the present invention, step 301 is implemented with the percentage weight or volume of the organic materials are all equal and is 1:1:1:1:1:1:1. As non-limiting example of step 301, if garlic's weight is 100 grams, ginger's, onion's, garlangal root's, bell pepper's, turmeric's, and raddish's should also be 100 grams. It is noted that other measurement ratios such as percentage volume (% v/v) are within the scope of the present invention.

The list of some examples of the selected medicinal plants with antimonial properties according to step 301 is shown in Table 1 below.

TABLE 1
Some Examples of Medicinal Plants with Antimicrobial Properties

| | | Perentage of Weight or Volume |
|---|---|---|
| No. | Ingredient | (% w/w) or (% v/v) |
| 1 | Garlic | 1 |
| 2 | Ginger | 1 |
| 3 | Onion | 1 |
| 4 | Garlangal Root | 1 |
| 5 | Bell Peppers | 1 |
| 6 | Turmeric | 1 |
| 7 | Raddish | 1 |

Next, at step 302, the organic materials with antibiotic properties of step 301 are juiced together. In many aspects of the present invention, step 302 is realized by commercial juicer machines. All selected organic materials are juiced at the same time to obtain a liquid juice.

Next, at step 303, the juice is now mixed with apple cider vinegar (AVC) and powder spices at a second predetermined percentage relative to the total weight including the juice from step 302. In various aspect of the present invention, step 301 is implemented with powder spices are cinnamon, back peppers, and salt with the second predetermined percentage weight (% w/w) is 500:500:10:1:10. These spices all knowingly have health benefits. As non-limiting examples of the second predetermined weight, if the liquid juice's weight is 500 grams, AVC's weight is also 500 grams, cinnamon powder's is 10 g, black pepper's is 1 gram, and salt's is 10 grams. It is noted that other measurement ratios such as percentage volume (% v/v) are within the scope of the present invention. AVC is either Bragg® AVC, Vermont Village AVC, or any equivalent AVC available in the market.

TABLE 2
Components of the Antibiotics and Their Measurement Ratio

| | | Predetermined Percentage Weight |
|---|---|---|
| | Ingredients | (% w/w) or (% v/v) |
| 1 | Liquid Juice | 500 |
| 2 | AVC | 500 |
| 3 | Cinnamon | 10 |
| 4 | Black Pepper | 1 |
| 5 | Salt | 10 |

At step 304, the mixture from step 303 is stirred and simultaneously undergone anoxic fermentation in a condition absent of sunlight, for a predetermined time period, and at a predetermined temperature, and simultaneously stirred (agitated) at a predetermined speed. Anoxic fermentation is anaerobic pathway to break down glucose. In a condition without oxygen, anaerobic microorganism within the mixture will cause the mixture to ferment, i.e., breaking down glucose. In many aspects of the present invention, step 304 is implemented by placing the mixture of step 303 into a bioreactor. In the industrial scale, a fermentator is used for large quantity production. In some embodiments of the present invention, a stirred tank bioreactor (STBR) is used to implement step 304. In addition, the bioreactor containing the mixture is placed in a dark environment in the absence of sunlight. In some aspects of the present invention, the mixture of step 303 is allowed to anoxic ferment and stirred at a speed of 50 rpm for 7 days and in a temperature between 27° C. to 30° C.

The process 300 disclosed above achieves the following objectives:

Gaining in Bio-Availability.

Gaining in effectiveness against different groups of bacteria and fungi (that is larger DIZ).

Gaining in polyphenols having the capability of preventing bacteria from developing antimicrobial resistance (AMR).

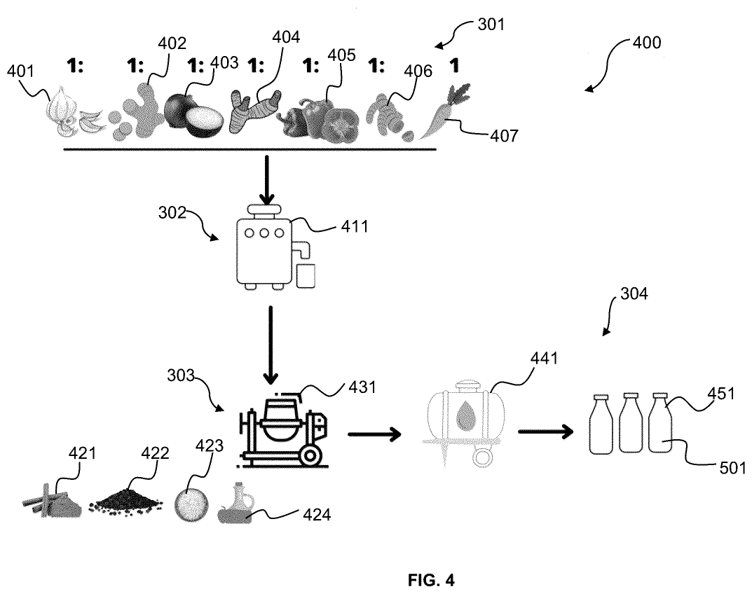

Next referring to FIG. 4, a diagram 400 illustrating the process of making the antibiotic of the present invention is presented. In the implementation of step 301, a garlic 401 having a predetermined first percentage weight or volume (% w/w or % v/v); a ginger 402 having a predetermined second percentage weight or volume (% w/w or % v/v); an onion 403 having a predetermined third percentage weight or volume (% w/w or % v/v); a garlangal root 404 having a predetermined fourth percentage weight or volume (% w/w or % v/v); a belly pepper juice 405 having a predetermined fifth percentage weight or volume (% w/w or % v/v); a turmeric 406 having a predetermined sixth percentage weight or volume (% w/w or % v/v); and a raddish 407 having a predetermined seventh percentage weight or volume (% w/w or % v/v) are selected. In one aspect of the present invention, step 301 is realized with the percentage weight of the organic materials are all equal and 1:1:1:1:1:1:1.

In the implementation of step 302, a juicer machine 411 is used. Juicer machine 411 can be a home scale for home use or industrial scale for commercial use. Home scale juicer machine 411 is a centrifugal juicer operating at 110V, 60 Hz, 370W, 3700 rotary speed to obtain the juice outlet diameter is 0.7 inch. Industrial juicer machine can be a screw press cold juice presser commonly available in the market.

In the implementation of step 303, a mixer 431 is used to mix the juice of step 302 with apple cider vinegar (ACV) and powder spices such as cinnamon, back peppers, and salt with the second predetermined percentage weight or volume (% w/w or % v/v) at 500:500:10:1:10. Mixer 431 can be the same as centrifugal juicer machine 411 or a blender.

Finally in the implementation of step 304, a bioreactor 441 is used to keep out oxygen molecules. Bioreactor 411 containing the mixture is placed in a dark environment absent of sunlight. The mixture of step 303 is allowed to ferment for 7 days and in a temperature between 27° C. to 30° C. In many embodiments of the present invention, bioreactor or a stirred tank bioreactor (STBR) 411 designed to stir the fermenting mixture at 50 rpm throughout the fermentation period of 7 days. Afterward, an antibiotic compound 501 of the present invention is obtained. Antibiotic compound 501 is stored in bottles 451 and shaked well before each use within one year. In the industrial scale, step 304 is implemented with a fermentator. In bioreactor, STBR, and fermentator 411, various fermentation parameters such as temperature, agitation speed, quantity of gas in and gas out are used.

### Experiment 1—Kirby-Bauer Test

The Kirby-Bauer test takes a pure bacterial culture and spreads it onto an agar plate. Then, a small disk infused with antibiotic compound 501 (appropriately called an antibiotic disk) is placed onto the agar plate. Various disks with different antibiotics are placed around the plate, and the bacteria are left to incubate for a certain amount of time. Once the disk is placed onto the plate, the antibiotics will start to diffuse out. If the bacteria being studied is sensitive to the antibiotic, then no bacteria will grow close to the disk because it will be killed by the medication. But farther away from the antibiotic disk, the concentration of the antibiotic decreases. At a certain distance from the disk, the bacterial grow again because the antibiotic concentration is too low to affect the bacteria. The area around the antibiotic disk that has no bacterial growth is known as the zone of inhibition (DIZ). The zone of inhibition is a uniformly circular zone of no bacterial growth around the antibiotic disk. The larger this zone is, the more sensitive the bacteria is to that antibiotic. The smaller the zone is, the more resistant (and, thus, less sensitive) the bacteria is. The Kirby-Bauer test was performed to compare fifth antibiotic sample 205 and antibiotic compound 501 of the present invention against various bacteria such as S. aureus, K. pneumonia, E. coli, P. aeruginosa, Salmonella sp., and Listeria monocytogenes.

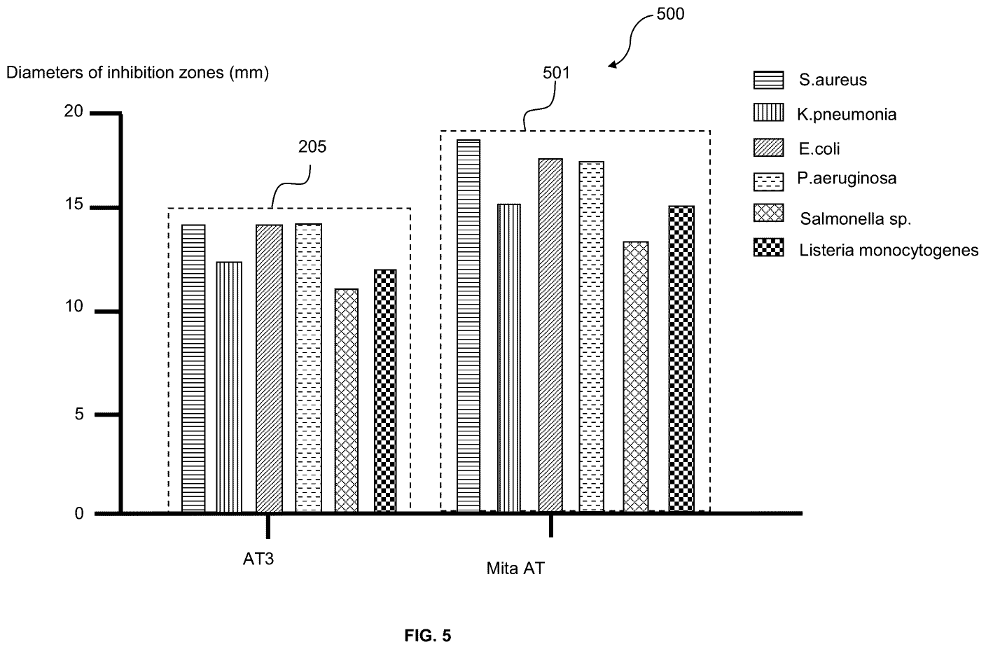

Next, referring to FIG. 5, a plot 500 of DIZ versus different antibiotic samples including antibiotic samples 205 and 501 is illustrated. Fifth antibiotic sample 205 have a DIZ about 14 mm against S. aureus, K. pneumonia, E. coli, P. aeruginosa, Salmonella sp., and Listeria monocytogenes. Antibiotic compound 501 has a DIZ much higher than 15 mm. Thus, from the above experiment, antibiotic compound 501 is more effective against bacteria and fungi than the prior-art antibiotic samples 201-205.

### Experiment 2—Curcumin High Pressure Liquid Chromotology (HPLC) Analyses

Curcumin is a polyphenolic compound extracted from rhizomes of Zingiberaceae and Araceae. It has multiple functions including antioxidant, anti-inflammatory, and antibacterial effects and has the advantages of being green pollution-free and natural and having no residue. Curcumin is chemically known as 1,7-bis(4-hydroxy-3methoxyphenyl)-1,6-heptadien-3,5-dione. It is a diarylheptanoid, which contains two aromatic O-methoxy phenolic groups that are connected by two α, β-unsaturated carbonyl groups, thus, this structure gives the curcumin more features such as antioxidant, antiobesity, anti-inflammatory, antidiabetic, cardioprotective, antimicrobial, immune-modulatory, anticancer activity, etc However, curcumin taken orally is poorly absorbed and rapidly metabolized and eliminated. Therefore, the potential of curcumin as a therapeutic agent is limited by its poor bioavailability. This means that curcumin is not very well absorbed when consumed by humans, and what is absorbed is rapidly broken down and gotten rid of by the body. Normally only around 3% of turmeric is curcumin, then a serious amount of turmeric needs to be absorbed in order to have some kind of effect.

Lactobacillus plantarum, used as starter culture to produce turmeric beverages, was isolated and screened from the turmeric rhizomes. Fermented turmeric beverages were evaluated for its antioxidant activity using 2-2-diphenyl-1-picrylhydrazyl (DPPH) radical-scavenging activity and ferric-reducing antioxidant power (FRAP) assay. It is well known that a fermentative process resulted in an increase in antioxidant activity. The absorption of turmeric powder, turmeric powder-mixed encapsulated probiotic (TP) and encapsulated fermented turmeric beverage (TB) was measured in terms of antioxidant activity in the plasma. Plasma antioxidant concentration was higher when the administrated fermented turmeric beverage than other turmeric products, at all the time points. The maximum concentration (Cₘₐₓ) value and area under the plasma concentration versus time curve (AUC) were higher as compared to TB. The value was lower in the plasma with turmeric powder and TP. The results indicated that the fermentative turmeric possesses better bioavailability and in accordance with the concentrations of polyphenolic compounds in the rat plasma.

In addition, curcumin is proven to restore bacterial susceptibility to antibiotics by inhibiting its biofilm mode of growth and rendering it sensitive to antibiotics. Recently, its antibacterial action is found to be synergistic with several antibiotics. Polyphenols, a group of micronutrients in curcumin can modulate the microbiota and promote the growth of beneficial bacteria, all at the expense of pathogenic bacteria. In turn, a healthy gut microbiome is essential to the proper modulation of the immune system and the prevention of a broad range of common infections and illnesses. Simply put, the more curcumin component found in a sample, the harder for bacteria to form resistance. In addition, curcumin can work with other antibiotics to weaken bacteria and fungi. Thus, if the HPLC test results a higher curcumin content, the better the antibiotics.

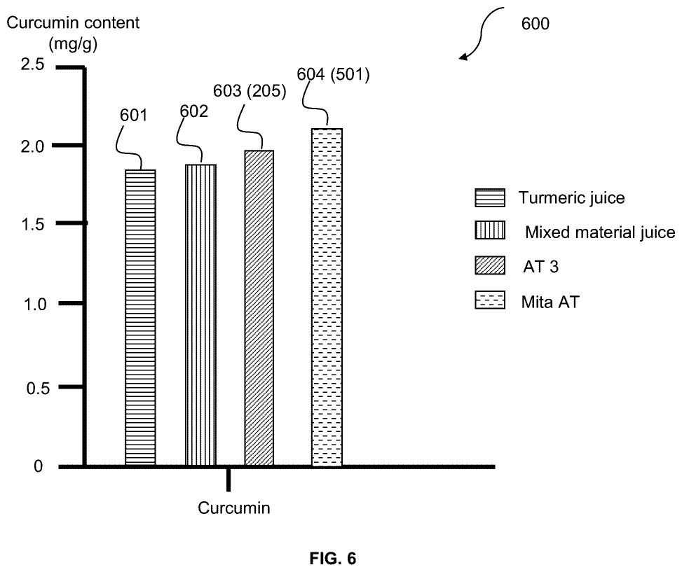

High-performance liquid chromatography (or High pressure liquid chromatography, HPLC) is a well-known analytical technique to separate and identify a certain components such as curcumin. Using this technique, it is also possible to identify, quantitate and collect curcumin. In the chromatography test, the compound under test such as antibiotic compound 501 is injected to be carried away by a mobile liquid. The mobile solution carries antibiotic compound 501 into a stationary liquid. Each component within antibiotic compound 501 binds to the molecules of the stationary liquid differently. This is called retention time t_{R} which is commonly used to identify peaks in a chromatogram. The retention time is characteristic of antibiotic compound 501. Therefore, retention times may be used to identify the compound by comparison with known quantity. In FIG. 6, four compounds are represented by four peaks separated in time in a comparative graph 600. These compounds include a turmeric juice 601, a mixed material juice 602 such as turmeric and garline, a fifth antibiotic sample 603 (the same as 205), and antibiotic compound 604 (the same as 501). Each elutes at a specific location, measured by the time elapsed between the moment of injection (time zero) and the time when the peak maximum elutes. By comparing each peak's retention time t_{R} with that of injected reference standards in the same mobile and stationary phase a chromatographer may be able to identify each compound.

Referring again to FIG. 6, graph 600 shows that antibiotic compound 604 (same as 501) has the highest curcumin content of more than 2 mg/g. While fifth antibiotic sample 603 (the same as 205) has about 2.0 mg/g, and mixed material juice 602 and turmeric juice 601 both have curcumin content lower than 2.0 mg/g. Thus, the HPLC test shows that antibiotic compound 501 of the present invention has the capability of preventing bacteria from developing antimicrobial resistance (AMR).

Food ingredients and nutrients such as thyme, mushrooms, ginger, garlic, sage, zinc, echinacea, elderberry, andrographis, saffron, estragon, allspice, oregano, rosemary, pelargonium or other medicinal herbs, plants, spices that have antimicrobial properties if developed in similar and analogous to method 300 above are within the scope of the present invention.

In addition, any foods, vegetables, tubers, peels, and plants such as banana, apple, pomegranate, sweet lime, orange, mango, oregano, garlic, parsley, sage, coriander, rosemary, and lemongrass), spices (cinnamon, clove), oils (citral) or organic compounds (vanillin), papaya, and the likes, if developed in similar and analogous to method 300 above are within the scope of the present invention.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

### REFERENCES

1. Leekha, Surbhi; Terrell, Christine L.; Edson, Randall S. (2011-02-01). “General principles of antimicrobial therapy”. Mayo Clinic Proceedings. 86 (2): 156-167.
2. Flowers, Christopher R.; Seidenfeld, Jerome; Bow, Eric J.; Karten, Clare; Gleason, Charise; Hawley, Douglas K.; Kuderer, Nicole M.; Langston, Amelia A.; Marr, Kieren A. (2013-02-20). “Antimicrobial prophylaxis and outpatient management of fever and neutropenia in adults treated for malignancy: American Society of Clinical Oncology clinical practice guideline”. Journal of Clinical Oncology: Official Journal of the American Society of Clinical Oncology. 31 (6): 794-810.

1. An antibiotic composition, comprising:
a garlic juice having a first percentage weight or volume (% w/w or % v/v);
a ginger juice having a second percentage weight or volume (% w/w or % v/v);
an onion juice having a third percentage weight or volume (% w/w or % v/v);
a galangal root juice having a fourth percentage weight or volume (% w/w or % v/v);
a belly pepper juice having a fifth percentage weight or volume (% w/w or % v/v);
a turmeric juice having a sixth percentage weight or volume (% w/w or % v/v);
a radish juice having a seventh percentage weight or volume (% w/w or % v/v);
an apple cider vinegar (ACV) having an eighth percentage weight or volume (% w/w or % v/v);
a black pepper powder having a ninth percentage weight or volume (% w/w or % v/v);
a cinnamon powder having a tenth percentage weight or volume (% w/w or % v/v); and
salt powder having an eleventh percentage weight or volume (% w/w or % v/v), all mixed together and fermented and stirred at the same time in an anoxic condition without being exposed to sunlight for a predetermined period of time, at a predetermined temperature, and at a predetermined stirring speed so that said antibiotic composition is characterized by having antimicrobial activities, curcumin content, and polyphenol bioavailability.

2. The antibiotic composition of claim 1 wherein said first percentage weight or volume (% w/w or % v/v), said second percentage weight or volume (% w/w or % v/v), said third percentage weight or volume (% w/w or % v/v), said fourth percentage weight or volume (% w/w or % v/v), said fifth percentage weight or volume (% w/w or % v/v), said sixth percentage weight or volume (% w/w or % v/v), and said seventh percentage weight or volume (% w/w or % v/v) are the equal.

3. The antibiotic composition of claim 2 wherein said eighth percentage weight or volume (% w/w or % v/v) is equal to a total weight of said first percentage weight or volume (% w/w or % v/v), said second percentage weight or volume (% w/w or % v/v), said third percentage weight or volume (% w/w or % v/v), said fourth percentage weight or volume (% w/w or % v/v), said fifth percentage weight or volume (% w/w or % v/v), said sixth percentage weight or volume (% w/w or % v/v), and said seventh percentage weight or volume (% w/w or % v/v).

4. The antibiotic composition of claim 2 wherein said ninth percentage weight or volume (% w/w or % v/v) equals to 1/50 of said eight percentage weight or volume (% w/w or % v/v) and said total weight of said first percentage weight or volume (% w/w or % v/v), said second percentage weight or volume (% w/w or % v/v), said third percentage weight or volume (% w/w or % v/v), said fourth percentage weight or volume (% w/w or % v/v), said fifth percentage weight or volume (% w/w or % v/v), said sixth percentage weight or volume (% w/w or % v/v), and said seventh percentage weight or volume (% w/w or % v/v).

5. The antibiotic composition of claim 3 wherein said tenth percentage weight or volume (% w/w or % v/v) equals to 1/500 of said eight percentage weight or volume (% w/w or % v/v) and said total weight of said first percentage weight or volume (% w/w or % v/v), said second percentage weight or volume (% w/w or % v/v), said third percentage weight or volume (% w/w or % v/v), said fourth percentage weight or volume (% w/w or % v/v), said fifth percentage weight or volume (% w/w or % v/v), said sixth percentage weight or volume (% w/w or % v/v), and said seventh percentage weight or volume (% w/w or % v/v).

6. The antibiotic composition of claim 2 wherein said eleventh percentage weight or volume (% w/w or % v/v) equals to 1/50 of said eight percentage weight or volume (% w/w or % v/v) and said total weight of said first percentage weight or volume (% w/w or % v/v), said second percentage weight or volume (% w/w or % v/v), said third percentage weight or volume (% w/w or % v/v), said fourth percentage weight or volume (% w/w or % v/v), said fifth percentage weight or volume (% w/w or % v/v), said sixth percentage weight or volume (% w/w or % v/v), and said seventh percentage weight or volume (% w/w or % v/v).

7. The antibiotic composition of claim 1 wherein said antimicrobial activities are characterized as having diameters of inhibition zone (DIZ) greater than 15 mm against Staphylococcus aureus (S. aureus), Klebsiella pneumonia (K. pneumonia), Escherichia Coli (E. Coli), Pseudomonas aeruginosa (P. aeruginosa), and Listeria monocytogenes (L. moncytogenes).

8. The antibiotic composition of claim 7 wherein said antimicrobial activities are characterized as having diameters of inhibition zone (DIZ) greater than 14.5 mm against Salmonella sp.

9. The antibiotic composition of claim 8 wherein said antimicrobial activities are characterized as having curcumin content greater than 2 mg/g.

10. The antibiotic composition of claim 8 wherein said predetermined period of time is 7 days and at said predetermined temperature is between 27° C. to 30° C.

11. A method of preparing an antibiotic tonic, comprising:
preparing antimicrobial fruits having equal first percentage weight or volume (% w/w or % v/v) of 1:1 to one another;
juicing said antimicrobial fruits to obtain antimicrobial fruit juice, wherein said antimicrobial fruits further comprise garlic, ginger, onion, garlangal root, belly pepper, turmeric, and radish;
mixing said antimicrobial fruit juice with a second solvent juice having equal second relative weight or volume (% w/w or % v/v) with said antimicrobial fruit juice and with a first powder spice and a second powder spice having a third relative percentage weight or volume (% w/w or % v/v) and with a third powder spice having a fourth percentage weight or volume (% w/w or % v/v) with respect to said antimicrobial fruit juice and said AVC to form a solution; and
anoxic fermenting and simultaneously stirring said solution in an absence of light condition for a predetermined period of time and for a predetermined temperature range to obtain said antimicrobial tonic characterized by having antimicrobial activities, curcumin content, and polyphenol bioavailability.

12. The method of claim 11 wherein said first powder is cinnamon powder, said second powder spice is salt, and said third powder spice is black pepper and said second solvent juice is apple cider vinegar (ACV).

13. A method of claim 11 wherein said garlic, ginger, onion, garlangal root, belly pepper, turmeric, and radish have said first percentage weight or volume (% w/w or % v/v) of 1:1:1:1:1:1:1.

14. The method of claim 11 wherein said second relative weight or volume (% w/w or % v/v) is 1:1.

15. The method of claim 12 wherein said third relative weight or volume (% w/w or % v/v) is 1/50.

16. The method of claim 14 wherein said fourth relative weight or volume (% w/w or % v/v) is 1/500.

17. The method of claim 11 wherein said antibiotic tonic is characterized as having diameters of inhibition zone (DIZ) greater than 15 mm against Staphylococcus aureus (S. aureus), Klebsiella pneumonia (K. pneumonia), Escherichia Coli (E. Co/i), Pseudomonas aeruginosa (P. aeruginosa), and Listeria monocytogenes (L. moncytogenes).

18. The method of claim 16 wherein said antibiotic tonic is characterized as having diameters of inhibition zone (DIZ) greater than 14.5 mm against Salmonella sp.

19. The method of claim 17 wherein said antibiotic tonic is characterized as having curcumin content greater than 2 mg/g.

20. The method of claim 17 wherein said predetermined period of time is 7 days and at said predetermined temperature is between 27° C. to 30° C.
